(19) United States
(12) Patent Application Publication
(10) Pub. No.: US 2016/0143752 A1
(43) Pub. Date: May 26, 2016

(54) PROSTHETIC SOCKET SUSPENSION DEVICE EMBODIMENTS

(71) Applicant: LIM INNOVATIONS, INC., San Francisco, CA (US)

(72) Inventors: Garrett Ray Hurley, San Francisco, CA (US); Jesse Robert WILLIAMS, San Francisco, CA (US)

(21) Appl. No.: 14/951,878

(22) Filed: Nov. 25, 2015

Related U.S. Application Data
(60) Provisional application No. 62/084,602, filed on Nov. 26, 2014.

(51) Int. Cl. A61F 2/80; A61F 2/78

(52) U.S. Cl. CPC A61F 2/80; A61F 2/7812; A61F 2210/0071; A61F 2002/7806; A61F 2002/7818; A61F 2002/805

(57) ABSTRACT

Embodiments of the technology include a prosthetic socket suspension device having a distal cup portion and a liner garment portion, these two portions being bonded together. In various embodiments, a distal cup may be disposed within a liner, a liner may be disposed within a distal cup, and a distal cup may be disposed between distally bifurcated internal and an external layers of a liner. Distal cup are sized and shaped to accommodate a distal portion of a residual limb of a patient. Distal cup embodiments may be formed from either a thermoplastic or thermoset plastic composition. Thermoplastic cup embodiments may be thermally reformed around the distal portion of the limb so as to create a highly conformal fit. Embodiments of the technology further include methods fabricating the prosthetic suspension device, and methods of thermally reforming the suspension device so as to fit a distal aspect of the residual limb of a patient.

### CROSS-REFERENCE TO RELATED PATENT APPLICATIONS

This application claims priority to U.S. Provisional Patent Application No. 62/084,602 of Hurley and Williams, entitled “PROSTHETIC SOCKET SUSPENSION DEVICE: A PROSTEHETIC SOCKET LINER WITHIN A THERMOPLASTIC SUPPORT CUP AND BONDED THERETO,” as filed on Nov. 26, 2014. The entirety of which is herein incorporated by reference.

This application is also related to U.S. patent application Ser. No. 14/310,147 of Hurley and Williams, entitled “PROSTHETIC SOCKET AND SOCKET LINER WITH MOISTURE MANAGEMENT CAPABILITY,” as filed on Jun. 20, 2014, and U.S. patent application Ser. No. 14/663,360 of Hurley et al., entitled “MODULAR PROSTHETIC SOCKET,” as filed on Mar. 19, 2015. The latter patent application (Ser. No. 14/663,360) claims priority to U.S. Provisional Patent Application No. 61/955,939, filed Mar. 20, 2014, entitled “THERMOPLASTIC-ELASTOMERIC COMPOSITE MATERIALS AND ARTICLES MADE THEREFROM,” and U.S. Provisional Patent Application 62/045,433, filed Sep. 3, 2014, entitled “MODULAR PROSTHETIC SOCKET.” All referenced patent applications are commonly owned, and all are hereby incorporated by reference in their entirety into the present patent application.

### TECHNICAL FIELD

The invention relates to a prosthetic socket system that includes a suspension device that retains the socket on the residual limb of the patient. More specifically, the invention relates to a prosthetic socket liner disposed within and bonded to a distal support cup.

### INCORPORATION BY REFERENCE

All publications and patent applications identified in this specification are herein incorporated by reference to the same extent as if each such individual publication or patent application were specifically and individually indicated to be so incorporated by reference.

### BACKGROUND

It is not enough that a prosthetic socket simply fit, however well, on the residual limb of a patient; it must also resist distal slippage, up and down pistoning, and rotation. In the prosthetic arts, resisting these forms of instability on the residual limb is referred to as “suspending” the socket. Suspension devices for prosthetic sockets that include a sock-like liner garment that fit over at least the distal portion of a residual limb are well known. These devices are typically formed from an air impermeable elastomer material such as silicone, and are configured to allow radial distension while being resistant to axial distension. They fit closely and conformally over the limb, particularly at their proximal end. The consistency of the silicone elastomer is friendly to the skin of the residual limb, and although it can easily be manually peeled away, it readily forms a substantially hermetic seal that effectively isolates the distal portion of the residual limb within the confines of the liner.

By virtue of this hermetic seal, the liner garment resists being pulled distally from the residual limb because of the vacuum created by a pulling of the liner from the surface of the limb. However, the seal is commonly imperfect, and some air can leak past the liner seal, and into the space between the liner and the surface of the residual limb. More significantly, the residual limb releases sweat into that interfacing space. Both of these processes can compromise the stability of the liner on the limb. Thus, it is also advantageous and known in the art to provide fluid exits from the liner, which may include valves and be activated by pumps that draw out fluid.

In addition to the attributes of prosthetic socket liners that contribute to the security of the socket on the limb, a variety of solutions have been developed that mechanically secure or stabilize the liner within the socket. Suspension, thus, is typically achieved by a layered approach, various suspension approaches each making a contribution. However, suspension is inherently difficult; the residual limb needs to be treated gently; damaging the skin on the residual limb is wholly intolerable for the patient. And generally the residual limb, itself, does not provide points of mechanical advantage that could contribute to suspension. Accordingly, suspension solutions continue to be sought, particularly solutions that are effective but simple. Simplicity in terms of mechanism, a low profile design, ease in operation, and ease in donning and removal are all desirable attributes.

### SUMMARY

Embodiments of the provided technology are directed to a prosthetic socket suspension device as well as methods of fabricating, and custom reshaping of the device to be appropriate for an individual patient. Suspension, in this sense, refers to the capability of a prosthetic socket to grasp onto the residual limb stably, without slippage, pistoning, or rotation.

Embodiments of the invention are directed to an integral prosthetic socket suspension device that includes a distal cup portion and a prosthetic liner garment portion that are bonded together. The prosthetic socket liner garment portion is sized and shaped to accommodate a distal portion of a residual limb of the patient; the garment has an internal surface, an external surface, an open proximal portion, and a substantially closed distal portion. The distal cup portion is sized and configured to reside within a distal portion of a prosthetic socket frame and to accommodate a distal portion of the residual limb of a patient; the distal cup includes an internal surface, an external surface, an open proximal portion, and a substantially closed distal portion. The distal cup of these embodiments is not bonded or permanently connected to the prosthetic socket frame. A bonding of the liner garment and the distal cup integrates the two portions into a singular device.

In some embodiments (Type A) of the integral prosthetic socket suspension device, a distal portion of the liner garment is disposed within the distal cup, the internal surface of the distal cup being bonded to the external surface of the distal portion of the liner garment.

In some embodiments (Type B) of the integral prosthetic socket suspension device, the distal cup is disposed within a distal portion of the liner garment, the external surface of the distal cup being bonded to the internal surface of the distal portion of the liner garment.

In some embodiments (Type C) of the integral prosthetic socket suspension device, a distal portion of the liner garment includes bifurcated inner layer and outer layers, each of the inner and outer layers having an internal surface and an external surface. The distal cup is disposed between the inner and outer layers of the liner garment; the internal surface of the distal cup is bonded to the external surface of the inner leaf of the liner garment and the external surface of the distal cup is bonded to the internal surface of the outer leaf of the liner garment.

In some embodiments of the integral prosthetic socket suspension device, the distal cup comprises a thermoplastic composition; in other embodiments, the distal cup is formed from a thermoset plastic composition. With regard to thermoplastic embodiments, the thermoplastic composition of the distal cup may have a reformable temperature of between about 125° F. and about 160° F. In some of these embodiments, the thermoplastic composition of the distal cup includes ethylene-vinyl acetate (EVA) copolymer and polycaprolactone (PCL), wherein EVA has between about 45% and about 55% of the composition by weight, wherein vinyl acetate has less than about 25% of the EVA by weight, and wherein polycaprolactone has about between about 45% and about 55% of the composition by weight. An in some of these embodiments that are formed of a thermoplastic composition, the distal cup is shaped to conform to the residual limb of the patient by way of a direct molding of the distal cup around a distal end of the residual limb. In some embodiments, the distal cup is a modular feature in that it may be selected from an inventory of distal cups, the inventory including units that vary in at least one characteristic consisting of size and shapes.

In some embodiments of the integral prosthetic socket suspension device, the proximal portion of the distal cup comprises proximally extending fingers separated by open spaces. This finger configuration contributes to the ability of the distal cup to conform to the distal end of the residual limb.

Some embodiments of the integral prosthetic socket suspension device, further include a layer of bonding material disposed within an interface between the liner garment and the distal cup.

Some embodiments of the integral prosthetic socket suspension device, further include an evacuation port positioned in the distal portion of the liner garment and the distal cup that provides an escape route for air and moisture to an external environment.

Some embodiments of the integral prosthetic socket suspension device, further include a prosthetic socket frame attachment mechanism disposed at the distal end of the prosthetic socket suspension device, the attachment mechanism configured to attach to a distal end of the prosthetic socket frame hosting the suspension device.

In some embodiments of the integral prosthetic socket suspension device, the liner garment comprises an elastomeric portion and a fluid transport substrate portion.

In some embodiments of the integral prosthetic socket suspension device, the liner garment comprises a composition of varying durometer, the durometer of a proximal portion of the liner garment being greater than the durometer of a distal portion of the liner garment.

In another aspect, embodiments of the invention are directed to a self-suspending prosthetic socket that includes a prosthetic socket frame and an embodiment of an integral prosthetic socket suspension device. The prosthetic socket frame has a proximally open internal cavity sized and configured to host a residual limb, the limb being supported integral or singular prosthetic socket suspension device.

The integral prosthetic socket suspension device includes the any of the various embodiments and features thereof, as summarized above. Briefly, it includes a distal cup sized and configured to reside within a distal portion of a prosthetic socket frame; the distal cup has an internal surface, an external surface, an open proximal portion, a substantially closed distal portion, and is formed from either a thermoplastic or a thermoset plastic composition. The liner garment is sized and shaped to accommodate a distal portion of a residual limb of a patient; it includes an internal surface, an external surface, an open proximal portion, and a substantially closed distal portion. The liner garment and the distal cup are bonded together to yield the integral prosthetic socket suspension device; the prosthetic socket frame and the integral prosthetic socket suspension device are configured to be mechanically connectable to each other.

In another aspect, embodiments of the invention are directed to method of thermally reforming a prosthetic suspension device to fit a distal aspect of a residual limb. As summarized above, the prosthetic socket suspension device includes a prosthetic socket liner garment and a distal cup bonded together, wherein (1) the distal cup is sized and configured to reside within a distal portion of a prosthetic socket, and comprises a thermoplastic composition with a reformable temperature of between about 125° F. and about 160° F., and wherein (2) the liner garment is sized and shaped to accommodate a distal portion of a residual limb of a patient. Embodiments of the method of thermally reforming the prosthetic suspension device include heating the distal cup to a sufficient temperature and for a sufficient duration that the cup becomes amenable to thermal reforming, applying the heated distal cup to surround a distal portion of the residual limb and manually compressing it so as to closely fit the distal portion of the residual limb. The method concludes by allowing the distal cup to cool in place on the residual limb for a sufficient time that the distal cup is stable in its reformed condition, and removing the prosthetic suspension device from the distal portion of the residual limb.

In another aspect, embodiments of the invention are directed to method of method of fabricating a prosthetic socket suspension device that includes providing a distal cup portion, providing a liner garment portion, and bonding the liner garment and the distal cup portions together. The provided distal cup is sized and configured to reside within a distal portion of a prosthetic socket includes an internal surface, an external surface, an open proximal portion, a substantially closed distal portion. Embodiments of the distal cup are formed either a from a thermoset composition or a thermoplastic composition with a reformable temperature of between about 125° F. and about 160° . The provided liner garment is sized and shaped to accommodate a distal portion of a residual limb of a patient; the garment includes an internal surface, an external surface, an open proximal portion, and a substantially closed distal portion.

In some of these embodiments, bonding the liner garment and the distal cup together comprises bonding the internal surface of the distal cup to the external surface of the distal portion of the liner garment.

In some of these embodiments, bonding the liner garment and the distal cup together comprises bonding the external surface of the distal cup to the internal surface of the distal portion of the liner garment.

In some of embodiments of the prosthetic socket suspension device, a distal portion of the liner garment has an inner layer and an outer layer, each of the inner and outer layers, respectively, having an internal surface and an external surface, the distal cup being disposed between the inner and outer layers of the liner garment. In such embodiments, bonding the liner garment and the distal cup together includes bonding the internal surface of the distal cup to the external surface of the inner leaf of the liner garment and bonding the external surface of the distal cup to the internal surface of the outer leaf of the liner garment. In some embodiments, this type of bonding is accomplished by way of an overmolding procedure in which an already formed distal cup is supported in place within a mold, and the liner composition is drawn into the mold, surrounding the distal cup.

### BRIEF DESCRIPTION OF THE FIGURES

FIG. 1 shows a cross sectional side view of a prosthetic socket device suspension device that includes a prosthetic socket liner and a distal cup that are bonded together, in one embodiment of Type A.

FIG. 2 shows a top perspective view of a prosthetic socket device suspension device, in one embodiment of Type A, which includes a liner portion and a distal cup portion, the two portions being bonded together.

FIG. 3 shows a top perspective view of an embodiment of a prosthetic socket suspension device, in one embodiment of Type A, which includes a liner and an alternative embodiment of a distal cup, the liner and the distal cup embodiment being bonded together; this device embodiment varies from that shown in FIGS. 1-2 by having a bulbous distal end configured to conform to a distal end of a bulbous limb, as is characteristic of knee disarticulation amputation which leaves an intact femoral condyle.

FIG. 4 shows a top perspective view of a prosthetic socket suspension device, in one embodiment of Type A, which includes a liner and an alternative embodiment of a distal cup, the liner and the distal cup embodiment being bonded together; this particular embodiment having a bulbous end, as the embodiment of FIG. 3, but the distal cup has a proximal end characterized by proximally extending finger like projections.

FIG. 5 shows a side cross sectional view of the residual limb of a patient as hosted within a prosthetic socket suspension device, the device including a distal cup disposed within the distal portion of a prosthetic socket liner, and bonded thereto, in one embodiment of Type B.

FIG. 6 shows a top perspective view of a prosthetic socket suspension device, in one embodiment of Type C, which includes a liner and an alternative embodiment of a distal cup, the liner and the distal cup embodiment being bonded together; this particular embodiment characterized by having the distal cup embedded within the liner, as shown by the dotted outline.

FIG. 7 shows a cross sectional view of the embodiment of a prosthetic socket suspension device, in one embodiment of Type C, that includes a liner and an embodiment of a distal cup with proximally extending fingers, as shown in FIG. 6.

FIG. 8A shows a side cross sectional view of a prosthetic socket suspension device that includes a distal cup disposed within the distal portion of a prosthetic socket liner, and bonded thereto, in one embodiment of Type C.

FIG. 8B shows a side view of a prosthetic socket suspension device that includes a distal cup disposed within the distal portion of a prosthetic socket liner, and bonded thereto, in one embodiment of Type C.

FIGS. 9A-9B show side views of a prosthetic socket suspension device that includes a distal cup disposed within the distal portion of a prosthetic socket liner, and bonded thereto, in one embodiment. FIG. 9A shows the device with a distal cup in a standard size and configuration; FIG. 9A shows the device after the distal cup has been thermally molded directly over the residual limb of a patient, thereby customizing the size and shape to conform to the patient's residual limb.

FIGS. 10A-10C compare cross sectional views of prosthetic suspension devices taken through the mid-distal portion of the device that includes the distal cup. FIG. 10A shows a cross sectional view of distal cup embodiment of Type A.

FIG. 10B shows a cross sectional view of distal cup embodiment of Type B.

FIG. 10C shows a cross sectional view of distal cup embodiment of Type C.

FIG. 11 shows a side perspective view of a prosthetic socket suspension device, in one embodiment, which includes a prosthetic socket liner and an alternative embodiment of a distal cup, the liner and the distal cup embodiment being bonded together; the suspension device is disposed within a strut-based prosthetic socket to which it is mechanically connected.

FIG. 12 shows a side cross sectional view of a prosthetic socket suspension device, in one embodiment, includes a prosthetic socket liner and an embodiment of a distal cup, the liner and the distal cup embodiment being bonded together; the device disposed within a conventional laminated prosthetic socket to which it is mechanically connected.

### DETAILED DESCRIPTION OF THE INVENTION

### Overview of Device Embodiments

The technology provided herein relates to a prosthetic socket suspension device having a prosthetic socket gel liner portion and a distal cup portion that are bonded together to form a single integral device. Device embodiments are sized and configured to serve as a suspension mechanism that retains a prosthetic socket on the residual limb of the wearer. Starting with the patient's limb and proceeding outward, a series of levels of adherences or attachments that retain the socket on the limb can be enumerated through three levels:
1. At a first level of attachment, a gel liner portion adheres to the limb surface by virtue of the closeness of the gel-to-skin contact circumferentially around the proximal aspect of the liner, the closeness largely precluding the leakage of air from the external environment into a substantially closed environment within the liner. Integrity of the gel-to-skin seal may be supported by at least by way of a 1-way valve within the liner that allows the escape of air and moisture to the external environment. A pump external to the liner and distal cup can facilitate escape of air and moisture that accumulates within interfacing space between the liner and the skin.
2. At a second level of attachment, the gel liner portion and the distal cup portions of the prosthetic suspension device are bonded together, forming an integral device.
3. At a third level of attachment, the prosthetic suspension device embodiment is mechanically connected to the prosthetic socket; this is a reversible connection. By this sequential three level attachment arrangement, the prosthetic socket is ultimately suspended from the patient's residual limb.

In addition to the features of embodiments of the gel liner portion of the suspension system that are described herein, embodiments may include any liner garment feature described in U.S. patent application Ser. No. 14/310,147 of Hurley and Williams, entitled “A prosthetic socket and socket liner with moisture management capability”, as filed on Jun. 20, 2014. In addition to the features of embodiments of the distal cup portion of the suspension system that are described herein, embodiments may include any distal cup feature described within U.S. Provisional Patent Application No. 62/045,433 of Hurley et al., entitled “Improvements for a modular prosthetic socket: soft goods arrangements, hardware, and a flexible inner liner” as filed on Sep. 3, 2014. Still further, aspects of a prosthetic socket liner garment such as those described in U.S. Provisional Patent Application No. 62/163,577 of Hurley et al., entitled “An integrated multi-material, multi-layer prosthetic socket liner garment”, as filed on May 19, 2015 and in a Provisional Patent Application of Hurley et al., entitled “An integrated multi-material, multi-layer prosthetic socket liner garment” that is being filed coincidentally with the present application.

Three basic embodiment types of suspension device for a prosthetic socket are provided herein, all of which include a prosthetic liner garment portion and a distal cup portion bonded together to form a single integrated device. Type A embodiments have a distal cup portion disposed within a liner garment portion. Type B embodiments have a liner garment with is distal portion disposed within a distal cup. Type C embodiments have liner garment that bifurcates in its distal portion into inner and outer leaves or layers, and a distal cup portion is disposed between such inner and outer layers. Some embodiments of a distal cup portion of the suspension device have a total surface area of greater than 45,000 mm². Suspension device embodiments of type A, B, and C embodiments each have particular advantages and features, as described in detail below.

Embodiments of the suspension device may be usefully applied to a wide variety of residual limb forms as defined by their amputation level. However, provided embodiments may be particularly appropriate for disarticulation amputations, such as upper extremity elbow or wrist disarticulations, and such as lower extremity knee or ankle disarticulations. Such disarticulation amputations typically provide a residual limb with a condyle at its distal end. A condyle is adapted for bearing weight, unlike a mid-bone amputation, and can bear weight, within limits, without creating discomfort or pain for the individual. Further, a condyle typically takes a bulbous form, a distal wide point that narrows proximally.

The features of embodiments of the suspension device provided herein are compatible with the features of the distal end of the bone in that the suspension device is typically donned by pushing it over the distal end of a residual limb, and continuing to push in the proximal direction until the device is seated. This approach to donning contrasts with conventional gel liners, which are typically donned by everting or reflecting them to expose their internal distal surface, contacting the distal end of the residual limb, and rolling the liner on to the residual limb. While the push on approach would not be appropriate for limbs with a mid-bone amputation site that may have a sensitive distal end, the push-on approach to donning is a quick and suitable procedure for a residual limb with a condylar end.

Further, embodiments of the suspension device provided herein commonly include a distal cup having a wide distal end and a proximal facing cavity that narrows slightly at its proximal end, thus complementing a residual limb with bulbous distal end, a common form for a condylar feature. A well-fitting distal cup, complementary in shape to a bulbous end, is advantageous for suspending a prosthetic device by virtue of the grasping nature of the fit of the suspension device on the distal end of the limb. Further, and related to the grasping nature of the fit and push on application, embodiments of prosthetic suspension devices provided herein, the liner portion of these devices may be relatively short in comparison to conventional roll on liners. Some liner embodiments may, for example, embrace only about a third of the length of the residual limb that is disposed within a prosthetic socket, whereas conventional roll on liners typically embrace nearly the entire length of the residual limb within the socket.

### Type A Liner-Cup Embodiments (Liner Inside Distal Cup)

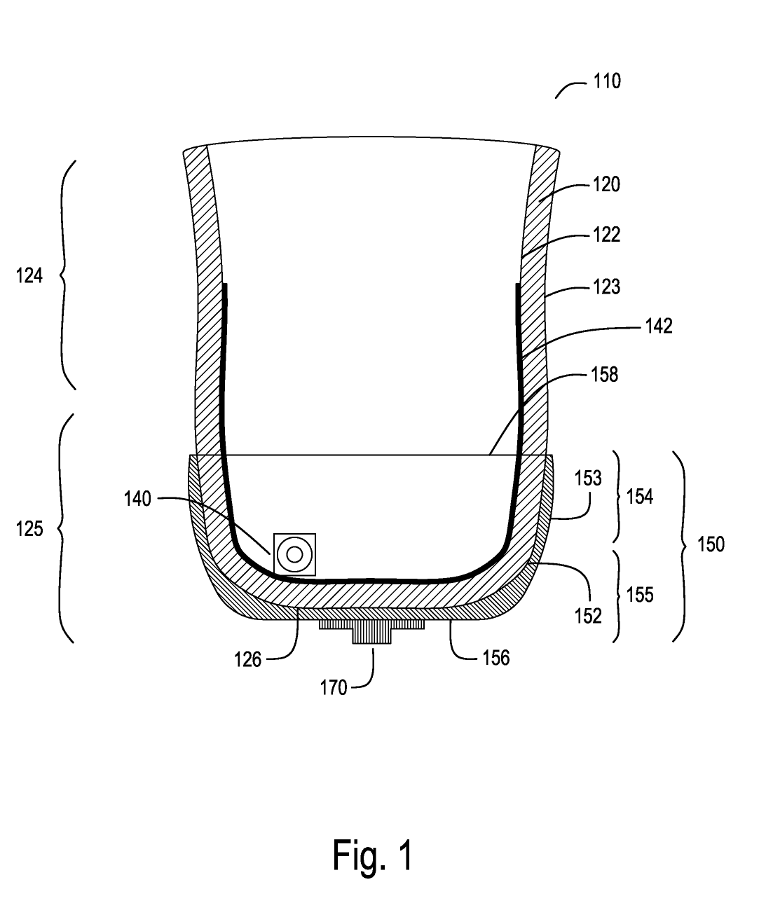

FIGS. 1-4 provide various depictions of a novel prosthetic suspension device of Type A (a liner garment within and bonded to an external distal cup). Various features of the provided suspension device embodiments are shown in these depicted embodiments; it is intended and included within the scope of the provided technology that any feature depicted or described in the context of any one embodiment or any one figure may be associated with any other provided embodiment.

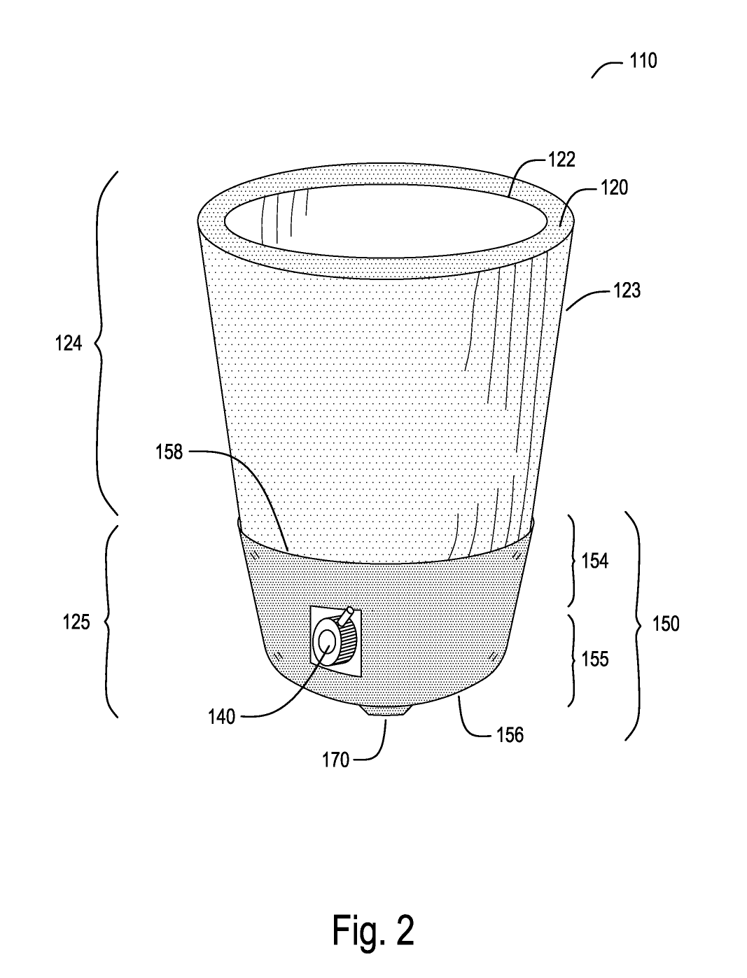

FIGS. 1 and 2 show a cross sectional side view and an upper perspective view, respectively, of an embodiment of a prosthetic device suspension device or supportive prosthetic socket liner 110 that includes a liner garment portion 120 disposed within a thermoplastic distal cup portion 150, the liner garment and distal cup being bonded together. The liner garment embodiment 120 has an internal surface 122, an external surface 123, a proximal portion 124, a distal portion 125, and a distal end 126. Suspension device embodiments further include a gel or elastomeric portion, and may include a fluid transport substrate portion. Other features of suspension device 110 include a distal fluid exit port 140 that provides a fluid escape route from within the confines of liner garment 120, through distal cup 150, and into the external environment. Distal fluid exit port 140 may be disposed at any suitable location in the distal portion 125 of liner garment 120 and the corresponding site in distal cup 150. FIG. 1 also shows a wicking layer or sock 142 that may be optionally included as an internal layer or partial layer of liner garment 120. This wicking layer, as a sock, can be donned separately by a patient, and not be included as a component of liner garment 120.

In some embodiments, proximal portion 124 of liner garment 120 is formed from a polymer composition having a relatively firm durometer (e.g., durometer of 10-20), which provides sufficient structural integrity that the proximal portion does not buckle when being applied to the residual limb in a push on manner, as described herein. Distal portion 125 of liner garment 120, in some embodiments, may have a relatively low durometer (e.g., durometer of 4-9) that provides a softer cushioning against the distal portion of the residual limb. The lower durometer composition is further suitable because the distal portion 124 of liner garment 120 is supported by the higher durometer distal cup 150, to which it is bonded.

Thermoplastic distal cup embodiment 150 has an internal surface 152, an external surface 153, a proximal portion 154, a proximal circumferential edge 158, a distal portion 155, and a distal end 156. A layer of bonding material, such as a silicon adhesive, and a padding or filler material may be disposed between liner garment 120 and distal cup 150.

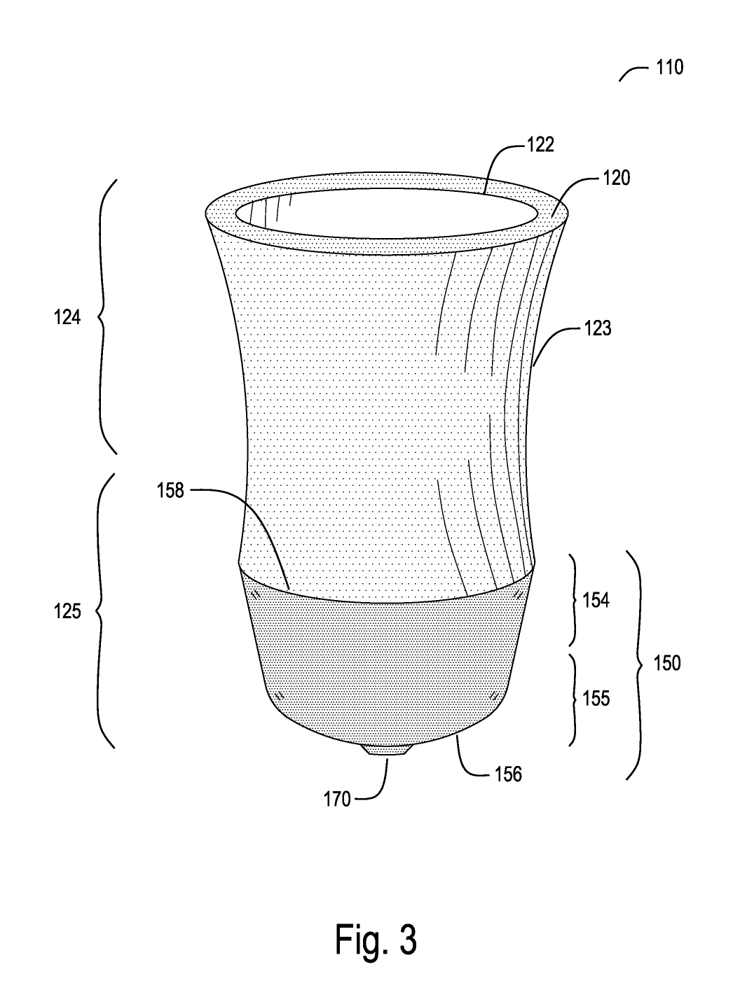

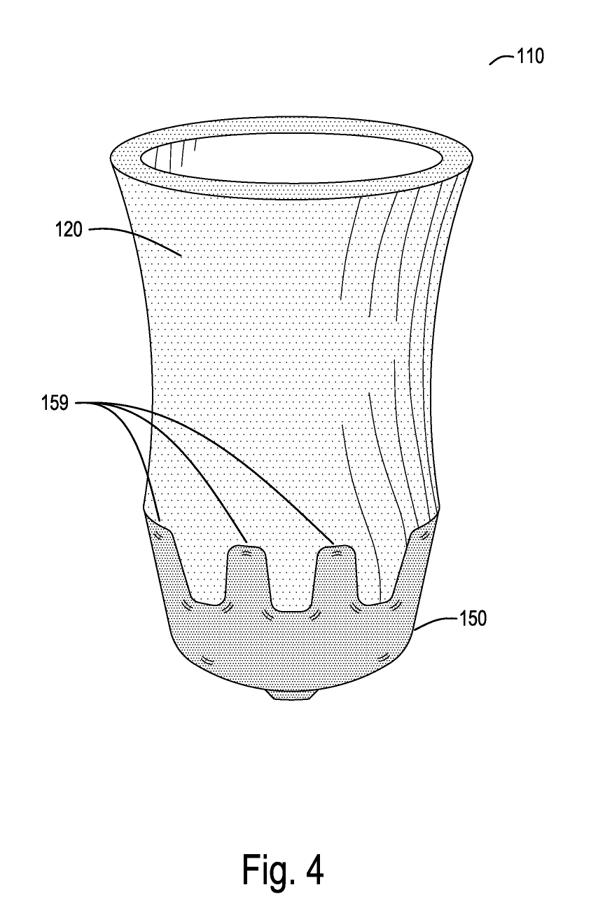

Related embodiments and variations of the embodiments shown in FIGS. 1-2 are described in detail below. Some particular embodiments of suspension device 110, for example, have a bulbous distal end, as defined primarily by the shape of distal cup 150. Bulbous embodiments are shown in FIGS. 3-4. Bulbous suspension device embodiments are not being given a separate label identifier in this disclosure because, in fact, shapes of the distal cup can vary in a continuum from straight cylindrical or even slightly tapering conical to a pronounced bulbous shape. Such variations may occur both in stock inventory shapes and/or may arise during a direct molding process over the distal end of a residual limb, as described below.

A bulbous distal end is particularly suitable for a knee-disarticulation amputation that leaves an intact condyle in place. A condyle provides a large distal end that is generally more suitable for bearing body weight than a typical above-knee or transfemoral amputation.

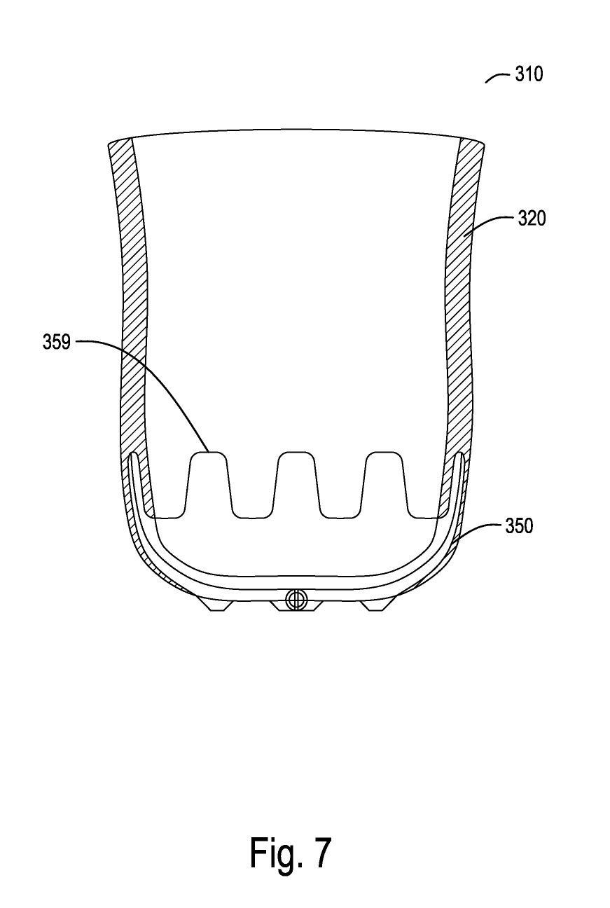

In another embodiment variation, FIG. 4 shows a suspension device 111 with a distal cup 150 that has a proximal edge 159 that is configured as a set up proximally extending fingers; this type of proximal edge is also seen in FIG. 7, depicting an embodiment of Type C. This fingered arrangement allows the distal cup to conform more closely to the shape of the distal end of a residual limb. In embodiments wherein the distal cup is formed from a thermoplastic material, such a higher degree of conformity can be achieved, in particular, by way of a process of direct molding of the distal cup around the distal end of the residual limb.

Any of the suspension device embodiments (Types A, B, and C) described herein may be provided in a range of sizes and shapes, such sizes and shapes referring either to the suspension device as a whole, or to its component portions, such as a liner garment or a distal cup. For example, a suspension device may be included in an inventory that includes, by way of example, three basic sizes (small, medium, and large). Shapes may also vary, as for example, a suspension device may be slightly tapering from proximal to distal end, it may be substantially straight from proximal to distal end, or it may be bulbous such that a distal portion is enlarged with respect to a more proximal location. Typically, shapes of a suspension device (as a whole) are largely determined by the shape of the distal cup portion. However, shapes of the liner garment portion may also vary in a manner similar to that of the distal cup.

Accordingly, embodiments of suspension device may be included in an inventory that can be drawn from in a modular manner when assembling a modular prosthetic socket, such as those described in U.S. patent application Ser. Nos. 13/675,761 and 14/213,788, wherein a complete prosthetic socket may be assembled from a selection of modular socket components. In addition to variation in any of size or shape that may be provided by suspension device components in an inventory, both size and shape (shape in particular) may be modified by a thermal reforming or direct molding method as applied to distal cup embodiments, by which such embodiments may be custom-fitted to a patient's residual limb.

Aspects of the thermoplastic composition, methods of making the composition and the distal cup, and thermal reforming of the distal cup to achieve a custom fit to the residual limb of a patient are described further below.

FIG. 3 shows an upper perspective view of an embodiment of a prosthetic device suspension 110 device that includes a liner portion 120 and an alternatively shaped a distal cup portion 150, the liner and the distal cup embodiment being bonded together; this device embodiment varies from that shown in FIGS. 1-2 by virtue of having a bulbous distal end configured to conform to a distal end of a bulbous limb as is characteristic of a knee disarticulation amputation that leaves an intact femoral condyle.

FIG. 4 shows an upper perspective view of an embodiment of a prosthetic socket suspension device 111 that includes a liner portion 120 and an alternative embodiment of a distal cup 51, the liner and the distal cup embodiment being bonded together. This particular suspension device embodiment has a bulbous end, as the embodiment of FIG. 3, but the distal cup 151 has a proximal end or edge characterized by proximally extending finger like projections 159.

FIG. 4 shows an upper perspective view of an embodiment of a prosthetic socket suspension device 111 that includes a liner 120 and an embodiment of a distal cup 151 that has a proximal edge arranged as proximally pointing fingers 159. The liner garment 120 and the distal cup embodiment 151 are bonded together, as in the embodiment shown in FIGS. 1-3, and other features of suspension devices 150 and 151 are substantially identical. As noted above, one of the linkages that maintains or suspends a prosthetic socket on a residual limb, per the suspension device embodiments described herein, is the bond between a liner garment embodiment and a distal cup embodiment. Liner garment embodiments 120 are highly elastic and conformable, and fit the residual limb easily and dynamically, as the limb shape subtly changes through the day or with any movement. Distal cup embodiments 150 and 151, in spite of having a degree of flexibility, in contrast, are harder and less elastic and compliant than the liner garment. Accordingly, it is desirable that the compliance of liner garment 120 not be substantially compromised by the relative non-compliance of distal cup embodiments 150 and 151.

However, the greater the amount of bonded interfacing area between liner garment 210 and distal cup embodiments 150 or 151, the more the compliance of the suspension device 110 or 111 will match that of the distal cup embodiment in the proximal portion of the device as a whole. Accordingly, the finger-like configuration 159 of the proximal edge of thermoplastic distal cup 151, provides a compromise between maximizing the bonded surface area between liner and cup by way of the finger-like projections 159, while allowing a compliant and elastic character to accommodate residual limb shape changes by way of the freely elastic and compliant portions of the liner garment 120 in the open space between the fingers.

### Type B Liner-Cup Embodiments (Distal Cup Inside Liner)

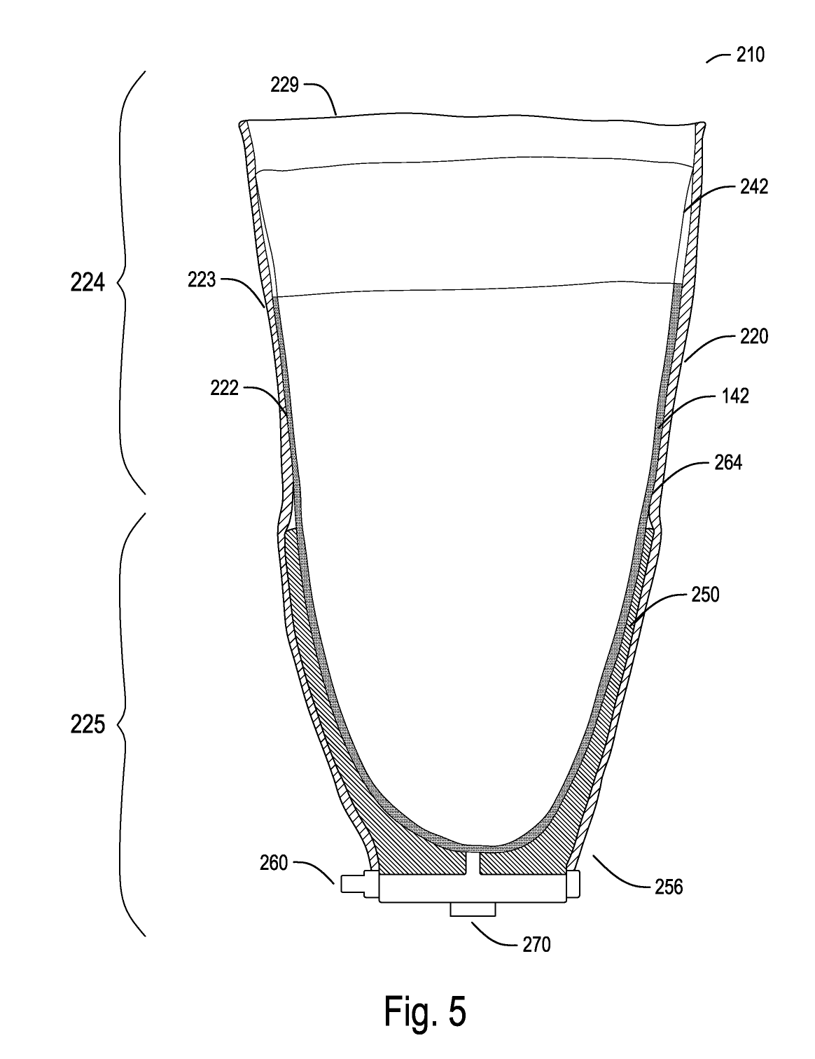

FIG. 5 shows a side cross sectional view of a prosthetic socket suspension device 210 of Type B that includes a distal cup 252 disposed within the distal portion 225 of a prosthetic socket liner garment 220, and bonded thereto, in one embodiment. FIG. 5, in addition to showing features of an embodiment of a suspension device 210, also shows a roll-on liner 242 that the patient is wearing, along with a wicking sock 142 that allows movement of air or moisture therethrough.

Prosthetic socket liner garment 220 has a proximal portion 224 with a proximal edge 229, a distal portion 225, an internal surface 222 and an external surface 223. Internal surface 222 typically includes a gel surface area, and further may include a wicking or fluid transport portion. These surface portions may be arranged in various advantageous configurations, as detailed in U.S. patent application Ser. No. 14/310,147 of Hurley and Williams.

The internal surface 222, comprising any of a gel composition or a fluid transport substrate portion, may extend through both proximal 224 and distal 225 portions of the liner garment. As described in detail above, in the context distal cup embodiments of Type A suspension device embodiments, distal cup 250 may be formed from a thermoplastic composition that allows thermal reforming of basic inventory models of a distal cup into one that is custom-fitted to each individual patient. Examples of thermoplastic composition and methods of making the composition are provided, as noted above, in U.S. patent application Ser. No. 14/644,630 of Hurley et al. Notably, such thermoplastic compositions become malleable and reformable at low temperature, and thus may be directly molded against the distal portion of a patient's residual limb such that the distal cup 250 substantially conforms to the size and shape of the residual limb.

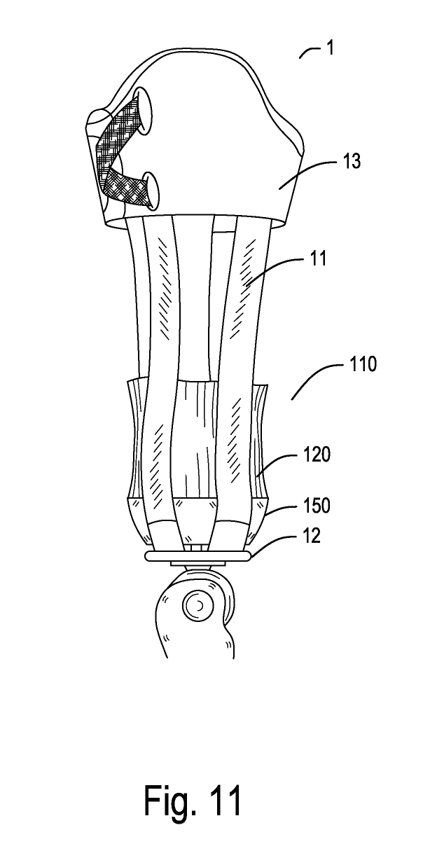

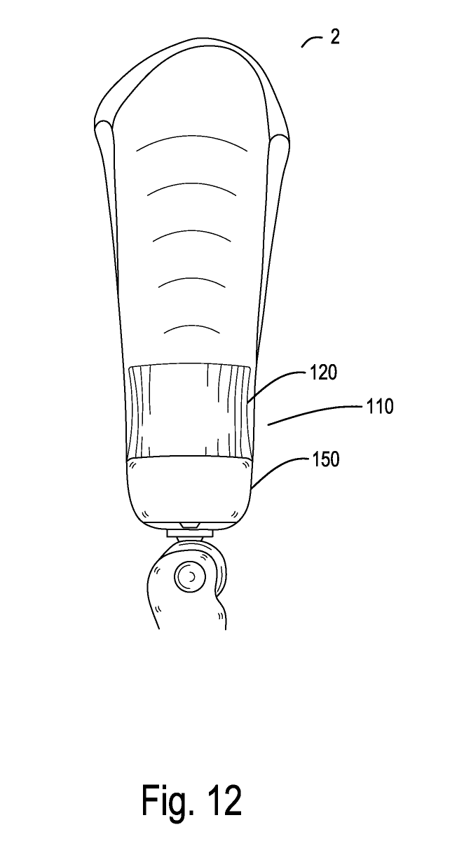

A generally horizontally oriented distal drain 260 is disposed at the distal end 256 of distal cup 250, the drain being configured to allow escape of perspiration liquid and/or air that accumulates within the liner; such fluid escape is typically facilitated by a vacuum draw. Also disposed at the distal end 256 of distal cup 250, and distally directed through liner garment 220 is attachment mechanism 270, configured to participate in the connection of suspension device 210 to a distal prosthetic element (not shown). Such attachment between device 210 and a distal prosthetic element is an important suspension feature by which a prosthetic device, as a whole, is stabilized on the residual limb of the patient (see FIGS. 11 and 12).

### Type C Liner-Cup Embodiments (Distal Cup Between Inner and Outer Leaves of Liner)

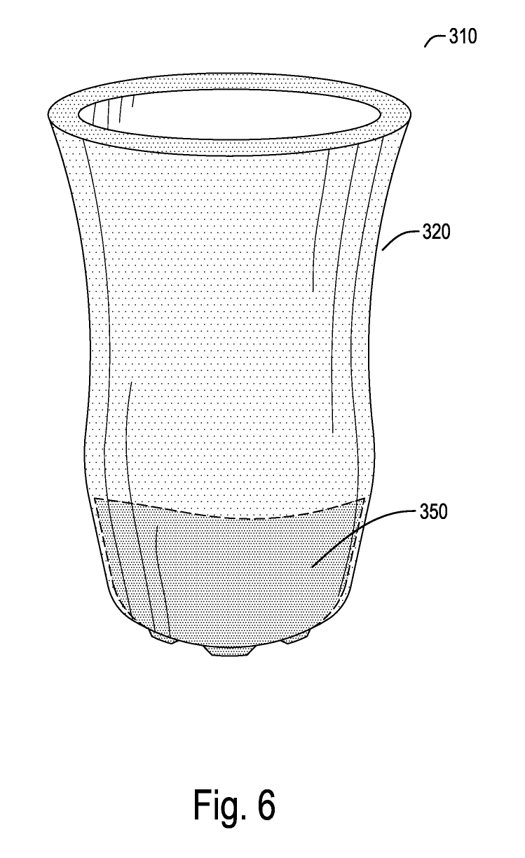

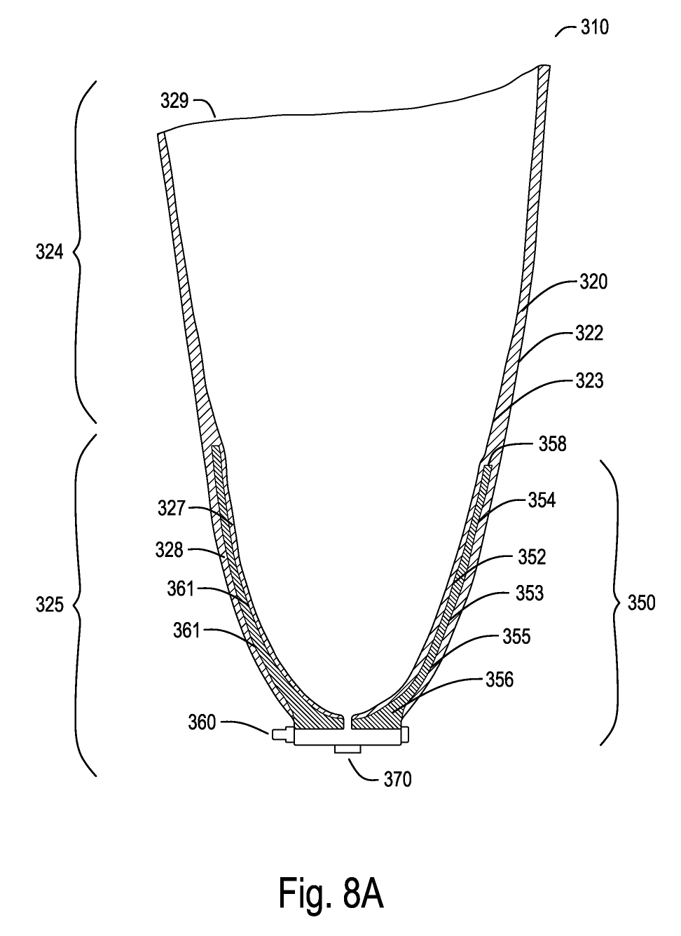

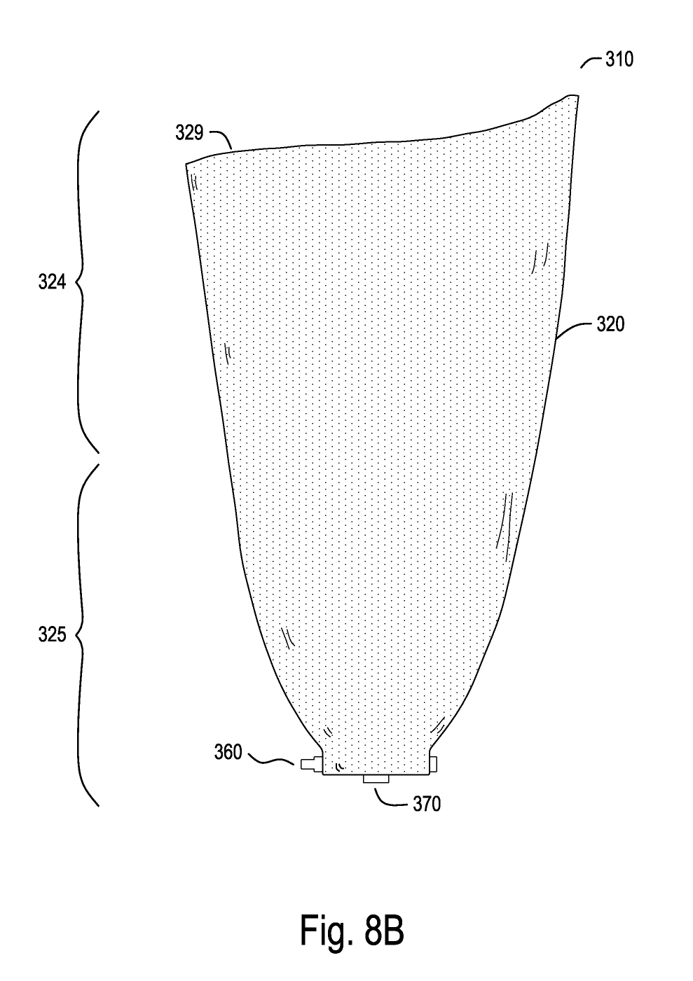

FIGS. 7-8B show prosthetic socket suspension device embodiments of Type C, wherein a distal cup is disposed between inner and outer distal leaves of a liner garment. FIG. 6 shows an upper perspective view of an embodiment of a prosthetic socket suspension device 310 that includes a liner portion 320 and an alternative embodiment of a distal cup 350, the liner and the distal cup embodiment being bonded together. This particular device embodiment is characterized by having the distal cup 350 disposed within an inner and outer layer of liner 320 and bonded thereto. Distal cup 350, being disposed within inner and outer layers of liner portion 350, is not visible from either an external or internal view, and thus is shown by the dotted outline.

FIG. 7 shows a cross sectional view of the embodiment of a prosthetic socket suspension device 310 like that of FIG. 6, but which includes features of embodiments shown in FIGS. 4 and 5. Suspension device 310 includes a liner portion 320 and an alternative embodiment of a distal cup 350 having finger like projections 359, the liner portion and the distal cup being integrated together so as to form a unified device.

FIGS. 8A and 8B show a side cross sectional view and side plane view, respectively, of a prosthetic socket suspension device 310 of Type C that includes a distal cup 352 disposed within the distal portion 325 of a prosthetic socket liner garment 320, and bonded thereto, in one embodiment. Socket liner garment 320 has a proximal portion 324 with a proximal edge 329, a distal portion 325 and a distal end 326. Liner garment 320 further has an internal surface 322 and an external surface 323. Internal surface 322 typically includes a gel surface area, and further may include a wicking or fluid transport portion. These surface portions may be arranged in various advantageous configurations, as detailed in U.S. patent application Ser. No. 14/310,147 of Hurley and Williams.

The internal surface 322, comprising any of a gel composition or a fluid transport substrate portion, includes both the proximal 324 and distal 325 portions of the liner garment. In its distal portion 325, the garment splits into an inner distal layer or leaf 327 and an outer distal layer 328; distal cup 350 is disposed between the inner 327 and outer 328 leaves, and bonded thereto with bonding material 361.

As described in detail elsewhere, distal cup 350 may be formed from a thermoplastic composition that allows thermal reforming of basic inventory models of a distal cup into one that is custom-fitted to each individual patient. Examples of thermoplastic composition and methods of making the composition are provided, as noted above, in U.S. patent application Ser. No. 14/644,630 of Hurley et al. Notably, such thermoplastic compositions become malleable and reformable at low temperature, and thus may be directly molded against the distal portion of a patient's residual limb such that the distal cup 350 substantially conforms to the size and shape of the residual limb.

Distal cup 350 includes an internal surface 352 and an external surface 353, and a proximal portion 354 with a proximal edge 358, a distal portion 355 with a distal end 356. The height of distal cup 250, as the cup is disposed generally within the distal portion of liner garment 320, corresponds approximately to the distal portion 325 of liner garment 350.

A generally horizontally oriented distal drain 360 is disposed at the distal end 356 of distal cup 350, the drain being configured to allow escape of perspiration fluid and/or air that accumulates within the liner, such escape is typically facilitated by a vacuum draw. Also disposed at the distal end 356 of distal cup 350, and distally directed through liner garment 320 is attachment mechanism 370, configured to connect device 310 to a distal prosthetic element. Such attachment between device 310 and a distal prosthetic element (not shown) is an important suspension element by which a prosthetic device, as a whole, is stabilized on the residual limb of the patient.

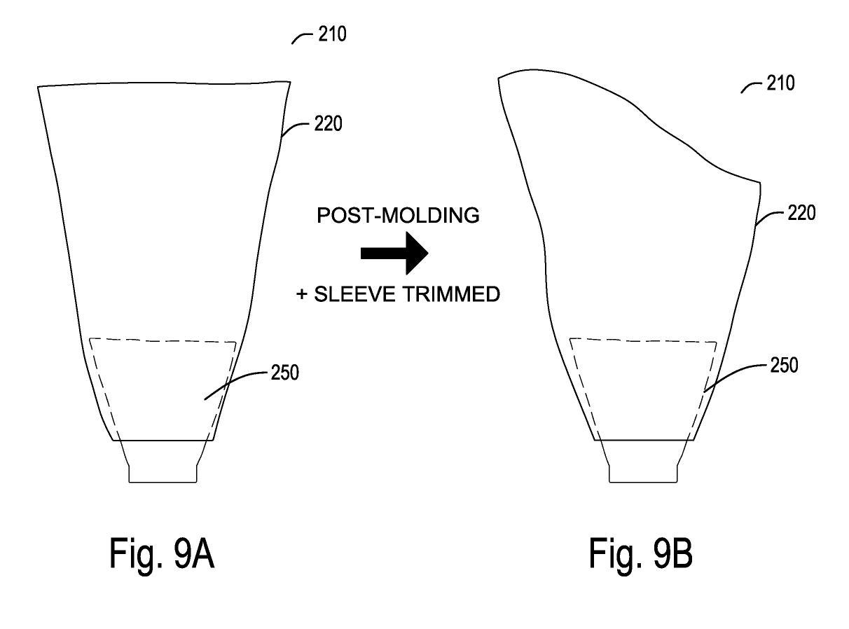

FIGS. 9A-9B show side views of a prosthetic socket suspension 210 device that includes a distal cup 250 disposed within the distal portion 225 of a prosthetic socket liner 220, and bonded thereto, in one embodiment. Although suspension device 210 is shown in FIGS. 9A-9B, these figures could equally apply to embodiments 110 and 310. FIG. 9A shows the suspension device with a distal cup in a standard size and configuration, as could be found in an inventory of devices. FIG. 9B shows the device after the distal cup has been thermally molded directly over the residual limb of a patient, thereby customizing the size and shape to conform to the patient's residual limb. Prosthetic socket suspension device 210, as seen in FIG. 9A may be understood be a freestanding device, as included in an inventory. Prosthetic suspension device 210, as seen in FIG. 9B, may be understood to represent the device as it would be seen in profile on the residual limb of a patient.

A “standard size and configuration” refers to one of a group of like suspension devices (110, 210, or 310) as contained in an inventory that can vary in size or shape. In one example, an array of sized suspension devices could include small, medium, and large device. In a second example, an array of differently shaped suspension devices could include tubular, conical, and bulbous shapes. In a third example that combines the attributes of the previous two, an inventory could include devices in nine models that vary both in size (three sizes) and shape (three shapes). These examples generally illustrate that a supportive prosthetic socket liner (including a liner and a distal cup bonded together) may be modular in character, i.e., varying in size and/or shape, but nevertheless having a common connection features such as attachment mechanism.

As described in Ser. No. 14/644,630 of Hurley et al., entitled “Improved modular prosthetic socket”, as filed on Mar. 19, 2015, a composite polymer composition comprising ethylenevinylacetate and polycaprolactone is thermally reformable at low temperature, and can be directly molded against a body portion. Thermal reforming allows a distal cup embodiment comprising such composition to expand elastically and conform around the distal end of a residual limb. Accordingly, it can be seen that distal cup 250, as seen in FIG. 9B is larger and has a different shape than that of distal cup 250 as seen in FIG. 9A.

The gel material of liner garment 220 is elastic and conformable, and thus the shape can expand or collapse to conform to the residual limb hosted within (as in FIG. 9B). Further, the material from which liner garment 220 is formed is soft and easily cut manually with a cutting implement such as scissors to accommodate the anatomy of the residual limb and body anatomy in proximity to the residual limb.

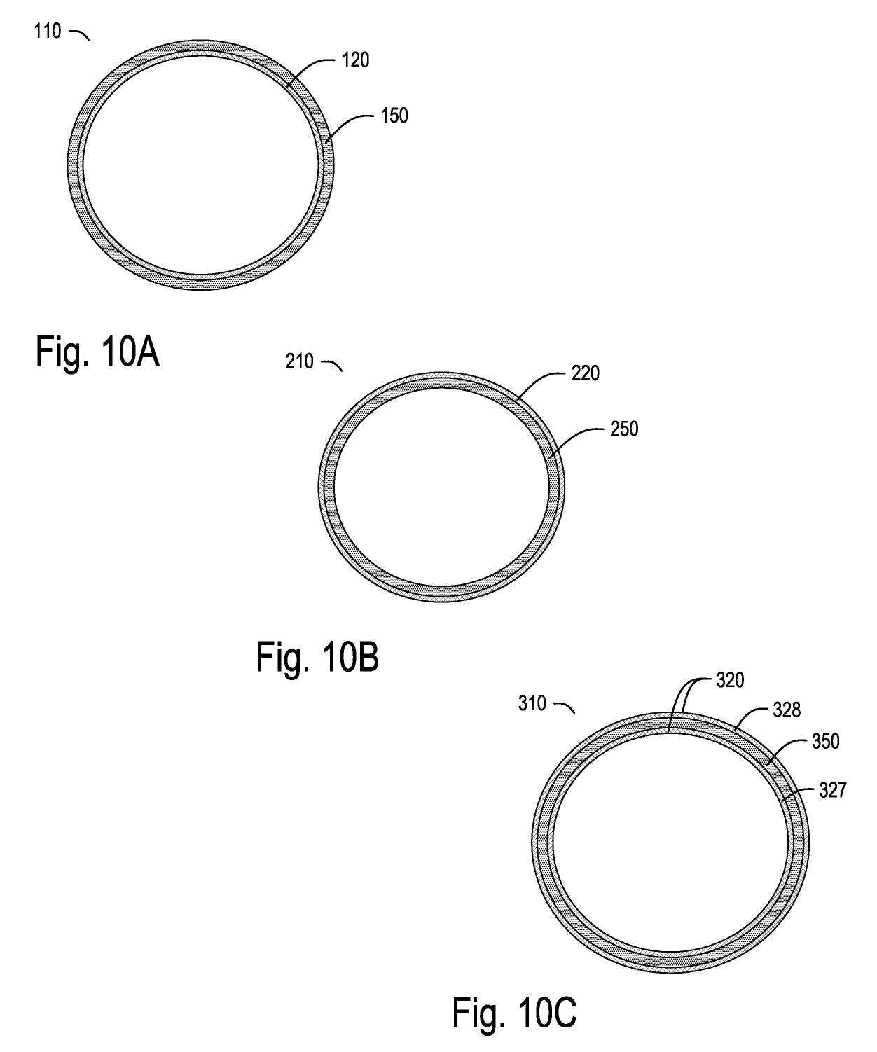

FIGS. 10A-10C compare cross sectional views of prosthetic suspension devices taken through the mid-distal portion of the device that includes the distal cup. FIG. 10A shows a cross sectional view of distal cup embodiment of Type A. In this embodiment of a prosthetic socket suspension device 110, prosthetic socket liner garment 120 surrounds distal prosthetic cup 150, the liner garment and distal cup being bonded together at their interface.

FIG. 10B shows a cross sectional view of distal cup embodiment of Type B. In this embodiment of a prosthetic socket suspension device 210, prosthetic socket liner garment 320 is disposed with distal prosthetic cup 250, the liner garment and distal cup being bonded together at their interface.

FIG. 10C shows a cross sectional view of distal cup embodiment of Type C. In this embodiment of a prosthetic socket suspension device 310, prosthetic socket liner garment 320, in its distal portion, is bifurcated into an inner layer 327 and an outer layer 328. Distal cup 350 is disposed between inner layer 327 and outer layer 328.

FIGS. 11 and 12 show embodiments of a prosthetic socket suspension device disposed within a strut-based modular prosthetic socket (FIG. 11) and a prior art laminated prosthetic socket (FIG. 12), respectively, in a demonstration of the application of the suspension device to different types of prosthetic socket frames.

FIG. 11 shows a side perspective view of an embodiment of a prosthetic socket suspension device 110 disposed within a strut based modular prosthetic socket 1, which includes modular struts 11, a modular distal base 12, and a modular brim element 13. Prosthetic socket suspension device 110 includes a prosthetic socket liner portion 120 and an embodiment of a distal cup 150, the prosthetic socket liner and the distal cup embodiment being bonded together. Suspension device 110 is disposed within a strut-based prosthetic socket 1 to which it may be mechanically connected. In another aspect, FIG. 11 may be appreciated showing as a self-suspending prosthetic socket system 1.

Examples of such a strut-based modular prosthetic socket 1 are described in U.S. patent application Ser. No. 13/675,761 (Pub. No. US 2013/0123940) of Hurley and Williams, entitled “Modular prosthetic sockets and methods for making same”, as filed on Nov. 13, 2012, in U.S. patent application Ser. No. 14/213,788 of Williams and Hurley, entitled “Modular prosthetic sockets and methods for making and using same”, as filed on Mar. 14, 2012, and in U.S. Provisional Patent Application No. 62/045,433 of Hurley et al., entitled “Improvements for a modular prosthetic socket: soft good arrangements, hardware, and a flexible inner liner” as filed on Sep. 3, 2014.

FIG. 12 shows a side cross sectional view of a prosthetic socket suspension device disposed 110 disposed within a prior art laminated prosthetic socket 2. Prosthetic socket suspension device 110 includes a prosthetic socket liner 120 and an embodiment of a distal cup 150, the liner and the distal cup embodiment being bonded together; the device disposed within a prior art laminated prosthetic socket 2 to which may be mechanically connected.

As noted above, moisture management features of liner garment 120 embodiments are described extensively in U.S. patent application Ser. No. 14/310,147 of Hurley and Williams. Liner garment embodiments 120 include an elastomeric portion, typically silicone-based and referred to as a silicone gel, and a fluid transport substrate that may take the form of a wicking surfaces or bulk liquid flow channels. These two portions can be configured in a number of different ways, however they all are directed toward providing an escape of moisture or air from within the confines of the liner garment to the external environment. Escape can take two routes: (1) a distal route that conveys moisture in liquid form toward the distal end of the liner and exit through a port, and/or (2) a lateral escape route through the garment liner to the external surface, wherein moisture can evaporate.

Accordingly, and as shown in U.S. patent application Ser. No. 14/310,147 (but not shown in herein) the elastomeric portion includes a substantially contiguous layer throughout liner garment 120. A fluid transport portion of liner garment may take any of several forms. For example, it maybe disposed on the internal surface and/or the external surface of liner garment 120. In some embodiments, the fluid transport portion 134 is disposed only within the distal portion of the liner garment 120.

In some embodiments, a fluid transport portion includes longitudinally aligned strips on the internal surface of the distal portion of the liner garment 120. In some embodiments, the fluid transport portion 134 includes longitudinally aligned strips on the internal surface of the distal portion of the liner garment 120.

In some embodiments, the fluid transport portion 134 includes any of a wicking fabric or a breathable mesh fabric In some embodiments, the internal surface 122 of the proximal portion 124 of the garment 120 includes only the elastomeric portion 132, the elastomeric internal surface being adapted to come into a sealing contact with a skin surface of the residual limb. Such sealing contact is circumferentially continuous around the residual limb and contributes to an ability of the suspension device to suspend the device on the residual limb.

Any of the thermoplastic distal cup embodiments described herein may be formed from a thermoplastic composition that allows thermal reforming of basic inventory models of a distal cup into one that is custom-fitted to each individual patient. Examples of thermoplastic composition and methods of making the composition are provided, as noted above, in U.S. patent application Ser. No. 14/644,630 of Hurley et al, which claims priority to U.S. Provisional Patent Application No. 62/045,433.

In one particular example of a thermoplastic composite composition, the composition includes (1) ethylene-vinyl acetate (EVA) copolymer and (2) polycaprolactone (PCL). (The composition as a whole can be referred to as EVA/PCL.) EVA accounts for between about 45% and about 55% of the composition by weight. The vinyl acetate portion of the EVA polymer accounts less than about 25% of the EVA by weight. The polycaprolactone accounts for between about 45% and about 55% of the composition by weight. Particular embodiments of the composition, as described, are monolithic, that is, the component polymer populations are well mixed, effectively forming a homogeneous composition. Embodiments of the EVA/PCL composition have a reformable or softening temperature of between about 125° F. and about 160° F., and a flexural modulus of about 3,500. In some particular embodiments of the composition, the molecular weight of the polycaprolactone (PCL) within the composition ranges between about 37,000 and about 80,000. In particular embodiments of the composition, the vinyl acetate proportion of the EVA comprises about 12% to about 28% of the EVA by weight. Accordingly, the weight ratio of PCL to EVA is in the range of about 0.43 to about 2.33. The weight/weight ratio of 0.43 represents a relative presence of about 43% PCL and about 57% EVA; the ratio of 2.33 represents a relative presence of about 70% PCL about 30% EVA.

Embodiments of the technology include a method of making a distal cup embodiment 150 or 151 include compounding pellets of ethylene-vinyl acetate (EVA) copolymer and pellets of polycaprolactone (PCL), wherein EVA comprises between about 45% and about 55% of the composition by weight, and wherein vinyl acetate comprises less than 25% of the EVA by weight, and wherein polycaprolactone comprises between about 45% and about 55% of the composition by weight. Such compounding method may include any one or more of heating, applying pressure, mixing, or applying pressure. Flowable composition may then be injected into an injection mold to create a distal cup of a desired size and form.

As noted above, and as provided in U.S. Provisional Patent Application No. 62/045,433, the thermoplastic composition of distal cup embodiments is amenable to thermal reforming such basic inventory models of a distal cup can be custom-fitted to each individual patient. Embodiments of a method of thermal reforming include heating a distal cup a sufficient temperature and for a sufficient duration that a composite polymeric material of the flexible support device becomes pliable; and applying sufficient and appropriately directed force to the pliable flexible support device such that the initial form of the distal cup embodiment changes toward a desired reformed shape.

In some embodiments, thermal reforming comprises heating the distal cup embodiment in a bath. In typical method embodiments, applying sufficient and appropriately directed force includes applying the pliable support device around a distal portion of the residual limb. This type of thermal reforming may be referred to as “direct molding”. In some embodiments, prior to applying the distal cup around a distal portion of the residual limb, the method includes wrapping the distal portion of the residual limb in a thermally insulating fabric. In typical and preferred embodiments, the desired reformed shape is substantially conformal to the distal portion of the residual limb.

Embodiments of the technology further include methods of assembling embodiments of the suspension device, thermally reshaping in a direct molding manner, and donning the device in a push-on manner.

A method of assembling a prosthetic suspension device includes providing a distal cup sized and configured to reside within a distal portion of a prosthetic socket, the distal cup as described above. The method further includes providing a liner garment sized and shaped to accommodate a distal portion of a residual limb of a patient, as described above. And the method concludes by bonding the liner garment into an internal aspect of the distal cup, so as to form an integrated or unitary device.

A method of thermally reforming a prosthetic suspension device (as summarized above) to fit a distal aspect of a residual limb includes (1) heating the distal cup to a sufficient temperature and for a sufficient duration that the cup becomes amenable to thermal reforming; and (2) applying the heated distal cup to surround a distal portion of the residual limb and manually compressing it so as to closely fit the distal portion of the residual limb; (3) allowing the distal cup to cool in place on the residual limb for a sufficient time that the distal cup is stable in its reformed condition; and (4) removing the prosthetic suspension device from the distal portion of the residual limb. This method of molding a thermally labile distal cup directly onto the distal end of a patient's residual limb is known as direct molding.

A method of donning a prosthetic suspension device (as summarized above) on a residual limb of a patient includes (1) placing the distal open portion of the distal cup and the liner garment portion proximate the distal open portion of the distal cup against a distal end of the residual limb; and (2) seating the suspension device onto the patient's residual limb in a push-on manner, i.e., by sufficient proximally directed force such that the distal cup slides over the distal end of the residual limb and is snugly fit around the distal end of the residual limb. This method of donning can be referred to as a push-on donning, and accordingly, the suspension device as a whole may be referred to as a push-on gel liner or push-on suspension device. This method of donning contrasts with the method of donning prior art gel liners, which are typically donned by way of a roll-on method, such gel liners thereby being referred to as roll-on gel liners.

Any one or more features of any embodiment of the invention, device or method, can be combined with any one or more other features of any other embodiment of the invention, without departing from the scope of the invention. It should also be understood that the invention is not limited to the embodiments that are described or depicted herein for purposes of exemplification, but is to be defined only by a fair reading of claims appended to the patent application, including the full range of equivalency to which each element thereof is entitled.

What is claimed is:

1. An integral prosthetic socket suspension device for a prosthetic socket frame, comprising:
a prosthetic socket liner garment sized and shaped to accommodate a distal portion of a residual limb of a patient, the garment comprising an internal surface, an external surface, an open proximal portion, and a substantially closed distal portion; and
a distal cup configured to reside within a distal portion of the prosthetic socket frame and to accommodate a distal portion of the residual limb of the patient, the distal cup comprising an internal surface, an external surface, an open proximal portion, and a substantially closed distal portion;
wherein the liner garment and the distal cup are bonded together to form the integral prosthetic socket suspension device, and
wherein the suspension device, when disposed within a prosthetic socket frame, is not permanently bonded thereto.

2. The integral prosthetic socket suspension device of claim 1, wherein a distal portion of the liner garment is disposed within the distal cup, the internal surface of the distal cup being bonded to the external surface of the distal portion of the liner garment.

3. The integral prosthetic socket suspension device of claim 1, wherein the distal cup is disposed within a distal portion of the liner garment, the external surface of the distal cup being bonded to the internal surface of the distal portion of the liner garment.

4. The integral prosthetic socket suspension device of claim 1, wherein a distal portion of the liner garment comprises an inner layer and an outer layer, each of the inner and outer layers having an internal surface and an external surface, wherein the distal cup is disposed between the inner and outer layers of the liner garment, and wherein the internal surface of the distal cup is bonded to the external surface of the inner leaf of the liner garment and the external surface of the distal cup is bonded to the internal surface of the outer leaf of the liner garment.

5. The integral prosthetic socket suspension device of claim 1, wherein the distal cup comprises a thermoplastic composition.

6. The integral prosthetic socket suspension device of claim 5, wherein the thermoplastic composition of the distal cup has a reformable temperature of between about 125° F. and about 160° F.

7. The integral prosthetic socket suspension device of claim 5, wherein the thermoplastic composition of the distal cup comprises ethylene-vinyl acetate (EVA) copolymer and polycaprolactone (PCL), wherein EVA comprises between about 45% and about 55% of the composition by weight, wherein vinyl acetate comprises less than about 25% of the EVA by weight, and wherein polycaprolactone comprises about between about 45% and about 55% of the composition by weight.

8. The integral prosthetic socket suspension device of claim 5, wherein the distal cup is shaped to conform to the residual limb of the patient by way of a direct molding of the distal cup around a distal end of the residual limb.

9. The integral prosthetic socket suspension device of claim 1, wherein the distal cup comprises a thermoset plastic composition.

10. The integral prosthetic socket suspension device of claim 1, wherein the distal cup is selected from an inventory of distal cups comprising variation in at least one characteristic consisting of size and shapes.

11. The integral prosthetic socket suspension device of claim 1, wherein the proximal portion of the distal cup comprises proximally extending fingers separated by open spaces.

12. The integral prosthetic socket suspension device of claim 1, further comprising a layer of bonding material disposed within an interface between the liner garment and the distal cup.

13. The integral prosthetic socket suspension device of claim 1, further comprising an evacuation port positioned in the distal portion of the liner garment and the distal cup that provides an escape route for air and moisture to an external environment.

14. The integral prosthetic socket suspension device of claim 1, further comprising a prosthetic socket frame attachment mechanism disposed at the distal end of the prosthetic socket suspension device, the attachment mechanism configured to attach to a distal end of the prosthetic socket frame hosting the suspension device.

15. The integral prosthetic socket suspension device of claim 1, wherein the liner garment comprises an elastomeric portion and a fluid transport substrate portion.

16. The integral prosthetic socket suspension device of claim 1, wherein the liner garment comprises a composition of varying durometer, the durometer of a proximal portion of the liner garment being greater than the durometer of a distal portion of the liner garment.

17. A self-suspending prosthetic socket, comprising:
a prosthetic socket frame comprising a proximally open internal cavity;
an integral prosthetic socket suspension device for a prosthetic socket frame, comprising:
a prosthetic socket liner garment sized and shaped to accommodate a distal portion of a residual limb of a patient, the garment comprising an internal surface, an external surface, an open proximal portion, and a substantially closed distal portion; and
a distal cup configured to reside within a distal portion of the prosthetic socket frame and to accommodate a distal portion of the residual limb of the patient, the distal cup comprising an internal surface, an external surface, an open proximal portion, and a substantially closed distal portion,
wherein the liner garment and the distal cup are bonded together to form the integral prosthetic socket suspension device; and
wherein the prosthetic socket frame and the integral prosthetic socket suspension device are configured to be mechanically connectable to each other.

18. A method of thermally reforming a prosthetic suspension device to fit a distal aspect of a residual limb, wherein the prosthetic socket suspension device comprises a prosthetic socket liner garment and a distal cup bonded together, wherein (1) the distal cup is sized and configured to reside within a distal portion of a prosthetic socket, and comprises a thermoplastic composition with a reformable temperature of between about 125° F. and about 160° F., and wherein (2) the liner garment is sized and shaped to accommodate a distal portion of a residual limb of a patient, the method comprising:
heating the distal cup to a sufficient temperature and for a sufficient duration that the cup becomes amenable to thermal reforming; and
applying the heated distal cup to surround a distal portion of the residual limb and manually compressing it so as to closely fit the distal portion of the residual limb;
allowing the distal cup to cool in place on the residual limb for a sufficient time that the distal cup is stable in its reformed condition; and
removing the prosthetic suspension device from the distal portion of the residual limb.

19. A method of fabricating a prosthetic socket suspension device comprising:
providing a distal cup sized and configured to reside within a distal portion of a prosthetic socket, the distal cup comprising an internal surface, an external surface, an open proximal portion, a substantially closed distal portion, and a thermoplastic composition with a reformable temperature of between about 125° F. and about 160°;
providing a liner garment sized and shaped to accommodate a distal portion of a residual limb of a patient, the garment comprising an internal surface, an external surface, an open proximal portion, and a substantially closed distal portion; and
bonding the liner garment and the distal cup together.

20. The method of fabricating a prosthetic socket suspension device claim 14, wherein bonding the liner garment and the distal cup together comprises bonding the internal surface of the distal cup to the external surface of the distal portion of the liner garment.

21. The method of fabricating a prosthetic socket suspension device claim 14, wherein bonding the liner garment and the distal cup together comprises bonding the external surface of the distal cup to the internal surface of the distal portion of the liner garment.

22. The method of fabricating a prosthetic socket suspension device claim 14, wherein a distal portion of the liner garment comprises an inner layer and an outer layer, each of the inner and outer layers, respectively, comprising an internal surface and an external surface, wherein the distal cup is disposed between the inner and outer layers of the liner garment, and wherein bonding the liner garment and the distal cup together comprises bonding the internal surface of the distal cup to the external surface of the inner leaf of the liner garment and bonding the external surface of the distal cup to the internal surface of the outer leaf of the liner garment.
